(19) United States
(12) Patent Application Publication
(10) Pub. No.: US 2023/0374597 A1
(43) Pub. Date: Nov. 23, 2023

(54) BIOMARKERS FOR PREDICTING OVERALL SURVIVAL IN RECORRENT/METASTATIC HEAD AND NECK SQUAMOUS CELL CARCINOMA

(71) Applicant: ASTRAZENECA AB, Sodertalje (SE)

(72) Inventors: Weimin LI, Wilmington, DE (US); Jiabu YE, Wilmington, DE (US); Han SI, Wilmington, DE (US); Nassim MORSLI, Cambridge (GB); Qu ZHANG, Cambridge (GB); Sophie WILDSMITH, Cambridge (GB)

(21) Appl. No.: 17/924,791

(22) PCT Filed: May 12, 2021
(86) PCT No.: PCT/EP2021/062707

§ 371 Date: Nov. 11, 2022

Related U.S. Application Data
(60) Provisional application No. 63/023,582, filed on May 12, 2020.
(60) Provisional application No. 63/031,238, filed on May 28, 2020.

(51) Int. Cl. C12Q 1/6886; G01N 33/574; C07K 16/28; A61P 35/04

(52) U.S. Cl. CPC C12Q 1/6886; G01N 33/57407; C07K 16/2827; C07K 16/2818; A61P 35/04; C12Q 2600/106; C12Q 2600/156; G01N 2333/70596; A61K 2039/507

(57) ABSTRACT

The present disclosure generally relates to methods for treating head and neck squamous cell carcinoma patients based on use of blood-based tumor mutation burden, PD-L1 expression, expression levels of immunomodulators, pro-angiogenesis markers and pro-inflammatory markers and/or identification of mutations in circulating tumor DNA.

This application is a U.S. national phase application under 35 U.S.C. 371 of International Patent Application No.: PCT/EP2021/062707, filed May 12, 2021, which claims the benefit of both U.S. Provisional Application No. 63/031,238, filed on May 28, 2020, and U.S. Provisional Application No. 63/023,582, filed on May 12, 2020, the disclosures of each of which are incorporated by reference herein in their entirety.

### FIELD OF THE DISCLOSURE

The present disclosure generally relates to methods for treating head and neck squamous cell carcinoma patients based on use of blood-based tumor mutation burden, PD-L1 expression, blood based markers, expression levels of immunomodulators, pro-angiogenesis markers and pro-inflammatory markers and/or identification of mutations in circulating tumor DNA.

### BACKGROUND OF THE DISCLOSURE

Recurrent/metastatic head and neck squamous cell carcinoma (R/M HNSCC) is a difficult cancer to treat. The standard of care (SoC) in the first-line setting is platinum-based doublet chemotherapy with cetuximab with limited survival benefits in general.

Immune checkpoint inhibitors have demonstrated clinical efficacy in the treatment of R/M HNSCC with anti-PD-1 blockade therapies and approved in first and second line settings. Durvalumab is an immune checkpoint inhibitor that blocks the interaction between programmed cell death ligand 1, or PD-L1, and its receptors. The cytotoxic activity of durvalumab has been found in various solid tumors leading to multiple approvals. Tremelimumab, is a cytotoxic T-lymphocyte—associated antigen 4, or anti—CTLA-4, monoclonal antibody. Since CTLA-4 and PD-L1/PD-1 pathways are largely non-redundant, combining them together could have additive effects and studies are ongoing to assess their clinical activities in different solid tumor types (see Burtness et al., The Lancet, Vol. 394, Issue 10212, P 1915-1928, 2019).

Despite the success of multiple anti-PD-L1 immune checkpoint inhibitors, it is worth noting that clinical response was restricted in a minority of patients with moderate improvement of overall survival, calling for efficient biomarkers to select patients most likely to benefit. Single arm or real-world evidence studies in R/M HNSCC have showed that tumor mutational burden (TMB), as measured in tumor tissue (tTMB), may be associated better with clinical outcomes with immune checkpoint inhibitor treatment. However, these studies failed to determine if TMB is predictive or prognostic and define clear predictivity cut-points for TMB.

### SUMMARY OF THE DISCLOSURE

The disclosure provides a method of predicting success of head and neck cancer treatment in a patient in need thereof, comprising determining the patient's tumor mutational burden (TMB), wherein a high TMB predicts success of treatment.

The disclosure further provides a method of treating head and neck cancer in a patient in need thereof, comprising: determining the patient's TMB, determining whether the TMB is high or low, and treating or continuing treatment if TMB is high or not treating or discontinuing treatment if TMB is low.

The disclosure further provides a method of treating head and neck cancer in a patient in need thereof, comprising: determining whether the patient has a somatic mutation in at least one of Lysine Methyltransferase 2D (KMT2D) gene or Ataxia-Telangiectasia Mutated (ATM) gene; and treating or continuing treatment if the patient has a somatic mutation in at least one of Lysine Methyltransferase 2D (KMT2D) gene or Ataxia-Telangiectasia Mutated (ATM) gene.

The disclosure further provides a method of predicting success of head and neck cancer treatment in a patient in need thereof, comprising determining PD-L1 expression in the patient's tumor cells and tumor-associated immune cells, wherein ≥50% of tumor cells express PD-L1 and/or ≥25% of tumor-associated immune cells express PD-L1 predicts success of treatment.

The disclosure further provides a method of treating head and neck cancer in a patient in need thereof, comprising: determining PD-L1 expression in the patient's tumor cells and tumor-associated immune cells; and treating or continuing treatment if ≥50% of the tumor cells express PD-L1 and/or ≥25% of the tumor-associated immune cells express PD-L1.

The disclosure further provides a method of predicting success of head and neck cancer treatment in a patient in need thereof, comprising determining levels of one or a plurality of protein biomarkers, wherein the protein biomarker is IL-23, osteocalcin, IL-6, neutrophil-to-lymphocyte ratio (NLR), von Willebrand factor (vWF), or Plasminogen activator inhibitor-1 (PAI-1); wherein an increased level of IL-23 or osteocalcin as compared to a reference level, and/or a decreased level of IL-6, NLR, vWF, or PAI-1 as compared to a reference level, and/or low tumor burden as compared to a reference level predicts success of treatment.

The disclosure further provides a method of treating head and neck cancer in a patient in need thereof, comprising: determining levels of one or a plurality of protein biomarkers, wherein the protein biomarker is IL-23, osteocalcin, IL-6, neutrophil-to-lymphocyte ratio (NLR), von Willebrand factor (vWF), or Plasminogen activator inhibitor-1 (PAI-1); and treating or continuing treatment if there is an increased level of IL-23 or osteocalcin as compared to a reference level, and/or a decreased level of IL-6, NLR, vWF, or PAI-1 as compared to a reference level, and/or low tumor burden as compared to a reference level.

### BRIEF DESCRIPTION OF THE DRAWINGS

FIG. 1A-1B show overall survival in all patients enrolled in the bTMB evaluable population sample collection period as compared with the biomarker evaluable populations in the EAGLE study.

FIGS. 2A-2C illustrate somatic single nucleotide variants (SNVs) or indels based on smoking status (FIG. 2A), PD-L1 expression (FIG. 2B), and HPV status (FIG. 2C) in the EAGLE study.

FIGS. 3A-3C show that blood TMB (bTMB) distributions across all three arms of treatment (durvalumab plus tremelimumab versus chemotherapy) were similar and independent of PD-L1 and HPV status in the EAGLE study.

FIG. 4 shows a forest plot illustrating TMB cut-points at greater than or equal to 16 mutations per megabase provided optimal improvement in overall survival for durvalumab versus chemotherapy for patients who have high blood TMB in the EAGLE study.

FIG. 5 shows a forest plot illustrating TMB cut-points at greater than or equal to 16 mutations per megabase provided optimal improvement in overall survival for durvalumab plus tremelimumab versus chemotherapy for patients who have high blood TMB in the EAGLE study.

FIG. 6 shows a forest plot illustrating TMB cut-points at greater than or equal to 16 mutations per megabase provided optimal improvement in progression-free survival for durvalumab versus chemotherapy for patients who have high blood TMB in the EAGLE study.

FIG. 7 shows a forest plot illustrating TMB cut-points at greater than or equal to 16 mutations per megabase provided optimal improvement in progression-free survival for durvalumab plus tremelimumab versus chemotherapy for patients who have high blood TMB in the EAGLE study.

FIG. 8 shows overall survival in EAGLE was improved with increasing blood TMB levels (in levels greater than or equal to 16 versus less than 16 mutations per megabase) for durvalumab and durvalumab plus tremelimumab treatment.

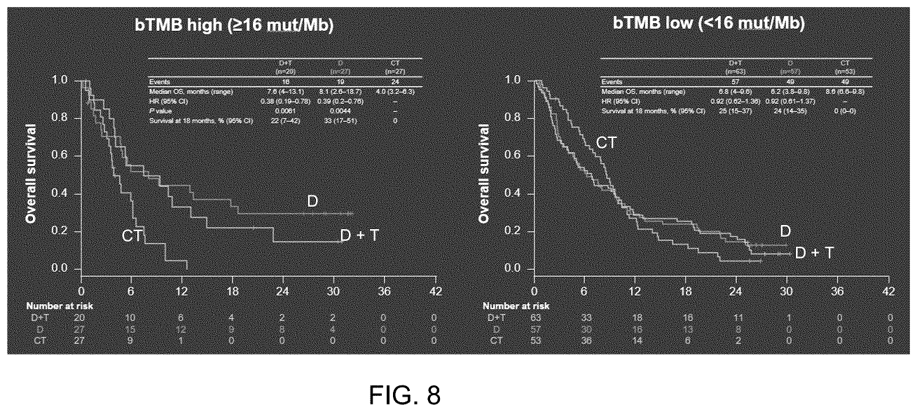

FIG. 9 shows progression-free survival in EAGLE was improved with increasing blood TMB levels (in levels greater than or equal to 16 versus less than 16 mutations per megabase) for durvalumab and durvalumab plus tremelimumab treatment.

FIG. 10 shows improved overall survival for durvalumab plus tremelimumab versus chemotherapy treated patients with mutations in KMT2D and ATM, with a hazard ratio of 0.39 (95% confidence interval: 0.17, 0.85) and 0.19 (95% confidence interval: 0.03, 1.03), respectively.

FIG. 11 shows a Kaplan Meier plot for overall survival for overlaid PD-L1 tumor cell (TC) subgroups for combined HAWK and CONDOR durvalumab monotherapy data. Data shows overlays of overall survival for TC subgroups (TC=0, TC≥1, TC≥10, TC≥25, TC≥50%).

FIG. 12 shows Kaplan Meier plots of overall survival between PD-L1 tumor cell subgroups for combined HAWK and CONDOR durvalumab monotherapy data. Data shows overall survival for PD-L1 TC subgroups (TC≥1, <1; TC≥10, <10; TC≥25, <25; TC≥50, <50%).

FIG. 13 shows a Kaplan Meier plot for overall survival for overlaid PD-L1 immune cell (IC) subgroups for combined HAWK and CONDOR durvalumab monotherapy data. The data shows overall survival for patients with immune cell scores of IC=0, IC>=1%, IC>=10, IC>=25, IC>=50.

FIG. 14 shows Kaplan Meier plots of overall survival between PD-L1 tumor immune cell subgroups for combined HAWK and CONDOR durvalumab monotherapy data. Data shows overall survival for PD-L1 IC subgroups (IC≥1, <1; IC≥10, <10; IC≥25, IC<25; IC≥50, IC<50%).

FIG. 15 shows Kaplan Meier plots of overall survival for PD-L1 TC50/IC subgroups for combined HAWK and CONDOR durvalumab monotherapy data.

FIG. 16 shows Kaplan Meier plots of overall survival between PD-L1 tumor immune cell subgroups for combined durvalumab monotherapy data.

FIG. 17 shows bootstrapped overall hazard ratio (HR) data for HAWK and CONDOR combined monotherapy durvalumab data (n=190 patients). Data shows overall survival (OS) HR [Biomarker +vs. Biomarker -] Unadjusted Cox PH (with Ties handling method=Effron) highlighting optimal cut-point of TC≥50 or IC≥25% with HR closest to 1.

FIG. 18 shows tissue TMB data availability from the HAWK and CONDOR studies.

FIG. 19 shows association of tissue TMB with smoking and HPV status in the HAWK and CONDOR studies.

FIG. 20 shows association of tissue TMB with overall survival in patients with low PD-L1 in the CONDOR studies.

FIG. 21 shows determination of the optimal TMB cut point using OS HR. Hawk and Condor with durvalumab and tremelimumab arms. N=126.

FIG. 22 shows association of low PD-L1 and low tissue TMB with overall survival in all evaluable patients in the HAWK and CONDOR studies.

FIG. 23 shows the association of neutrophil-to-lymphocyte ratio and tissue TMB with overall survival in the HAWK and CONDOR studies.

FIG. 24A-24C show comparison of observed and model simulated longitudinal tumor size (FIG. 24A), study dropout (FIG. 24B), and overall survival (FIG. 24C).

FIG. 25 shows the impact of baseline biomarkers on overall survival parameters.

FIG. 26 shows observed (solid lines) and model predicted (dotted lines) effects of serum cytokines on survival stratified by quartiles.

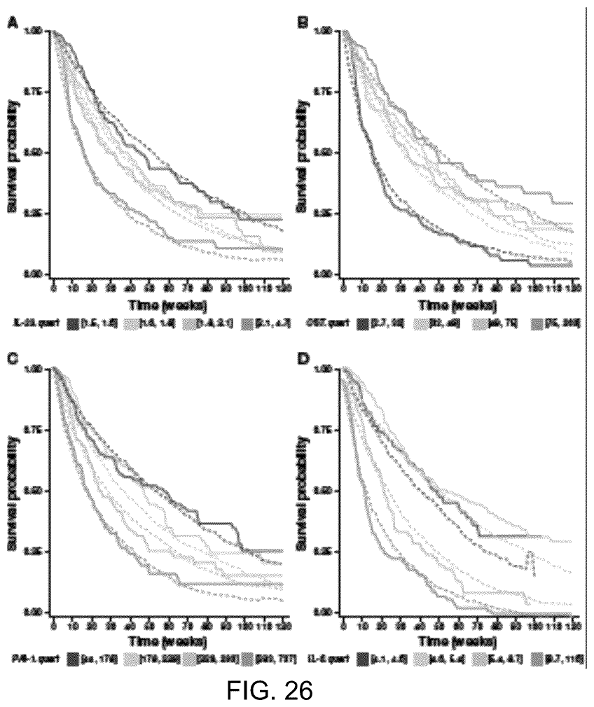

FIG. 27 shows all-comers subgroup by favorable (1)/unfavorable (0) biomarker profile (n=346). Median OS (n, 95% confidence interval [CI]) for the patients with favorable biomarker profile was 14.6 months (129, 11.2-21.4) versus 4.4 months (217, 3.6-5.3).

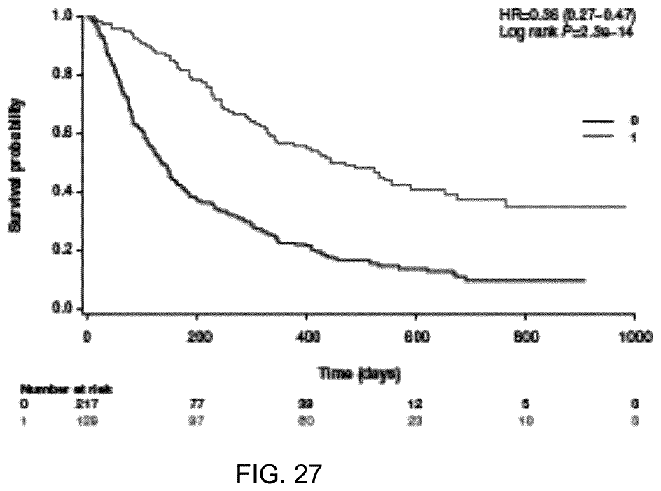

### DETAILED DESCRIPTION OF THE DISCLOSURE

The present disclosure generally relates to methods for treating head and neck squamous cell carcinoma patients based on use of blood-based tumor mutation burden, PD-L1 expression, expression levels of immunomodulators, pro-angiogenesis markers and pro-inflammatory markers and/or identification of mutations in circulating tumor DNA.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In some embodiments provided herein is method of predicting success of head and neck cancer treatment in a patient in need thereof, comprising determining the patient's tumor mutational burden (TMB), wherein a high TMB predicts success of treatment.

In some embodiments provided herein is a method of treating head and neck cancer in a patient in need thereof, comprising:
(a) determining the patient's TMB;
(b) determining whether the TMB is high or low; and
(c) treating or continuing treatment if TMB is high or not treating or discontinuing treatment if TMB is low.

“Tumor mutational burden” (TMB) refers to the quantity of mutations found in a tumor. TMB varies among different tumor types. Some tumor types have a higher rate of mutation than others. TMB can be measured by a variety of tools known in the field. In certain embodiments, these tools are the tumor whole exome sequencing. In some embodiments this sequencing can be measured using tools such as the Foundation Medicine and Guardant Health measurement tools. TMB can be determined through both blood and tissue measurements. Determining whether a tumor has high or low levels of tumor mutational burden can be determined by comparison to a reference population having similar tumors and determining median or mean level of expression. In some embodiments, a high TMB is defined as ≥12 to ≥20 mutations/megabase (mut/Mb). In some embodiments, a high TMB is defined as ≥16 mutations/megabase (mut/Mb). In some embodiments, a high TMB is defined as ≥20 mutations/megabase (mut/Mb).

In some embodiments, the patient has a lower neutrophil-to-lymphocyte ratio as compared to a reference level. Determining whether a patient has a lower neutrophil-to-lymphocyte ratio can be determined by comparison to a reference population having a similar cancer or tumor and determining the median or mean of the neutrophil-to-lymphocyte ratio. In some embodiments, a high TMB level and lower neutrophil-to-lymphocyte ratio are used as makers predictive of improved OS in patients receiving durvalumab and/or tremelimumab treatment.

In some embodiments, the patient has low expression of programmed death-ligand 1 (PD-L1) on tumor cells (TCs) and/or immune cells (ICs). In some embodiments, low expression is classified as ≤25% of the patient's tumor-associated immune cells express PD-L1 and ≤50% of the patient's tumor cells express PD-L1. In some embodiments, a high TMB level and low expression of PD-L1 are used as makers predictive of improved OS in patients receiving durvalumab and/or tremelimumab treatment.

In some embodiments, provided herein is a method of predicting success of head and neck cancer treatment in a patient in need thereof, comprising determining PD-L1 expression in the patient's tumor cells and tumor-associated immune cells, wherein ≥50% of tumor cells express PD-L1 and/or ≥25% of tumor-associated immune cells express PD-L1 predicts success of treatment.

In some embodiments, provided herein is a method of treating head and neck cancer in a patient in need thereof, comprising:
(a) determining PD-L1 expression in the patient's tumor cells and tumor-associated immune cells; and
(b) treating or continuing treatment if ≥50% of the tumor cells express PD-L1 and/or ≥25% of the tumor-associated immune cells express PD-L1.

In some embodiments, the success of treatment is determined by an increase in OS as compared to standard of care. In some embodiments, the success of treatment is determined by an increase in progression free survival as compared to standard of care. “Standard of care” (SoC) and “platinum-based chemotherapy” refer to chemotherapy treatment comprising at least one of methotrexate, docetaxel, paclitaxel, 5-FU, TS-1 or capecitabine.

As used herein, Overall Survival (OS) relates to the time-period beginning on the date of treatment until death due to any cause. OS may refer to overall survival within a period of time such as, for example, 12 months, 18 months, 24 months, and the like.

As used herein, Progression Free Survival (PFS) relates to the length of time during and after treatment that a patient lives with the head and neck cancer but the cancer does not get worse. PFS may refer to survival within a period of time such as, for example, 12 months, 18 months, 24 months, and the like.

In some embodiments, provided herein are methods of treating head and neck cancer in a patient in need thereof, comprising determining whether the patient has a somatic mutation in at least one of Lysine Methyltransferase 2D (KMT2D) gene or Ataxia-Telangiectasia Mutated (ATM) gene; and treating or continuing treatment if the patient has a somatic mutation in at least one of Lysine Methyltransferase 2D (KMT2D) gene or Ataxia-Telangiectasia Mutated (ATM) gene. In some embodiments, mutations in KMT2D and ATM are used as a biomarker predictive of improved OS in patients receiving durvalumab and/or tremelimumab treatment.

The term “KMT2D” encompasses “full-length” unprocessed KMT2D as well as any form of KMT2D that results from processing in the cell. The term also encompasses naturally occurring variants of KMT2D, e.g., splice variants or allelic variants.

The term “ATM” encompasses “full-length” unprocessed ATM as well as any form of ATM that results from processing in the cell. The term also encompasses naturally occurring variants of ATM, e.g., splice variants or allelic variants.

In some embodiments, provided herein is a method of predicting success of cancer treatment in a patient in need thereof, comprising determining levels of one or a plurality of protein biomarkers, wherein the protein biomarker is IL-23, osteocalcin, IL-6, neutrophil-to-lymphocyte ratio (NLR), von Willebrand factor (vWF), or plasminogen activator inhibitor-1 (PAI-1), wherein an increased level of IL-23 or osteocalcin as compared to a reference level, and/or a decreased level of IL-6, NLR, vWF, or PAI-1 as compared to a reference level, and/or low tumor burden as compared to a reference level predicts success of treatment. In some embodiments, IL-23, osteocalcin, IL-6, NLR, vWF, and PAI-1 are used as biomarkers predictive of improved OS in patients receiving durvalumab treatment.

In some embodiments provided herein is a method of treating head and neck cancer in a patient in need thereof, comprising:
(a) determining levels one or a plurality of protein biomarkers, wherein the protein biomarker is IL-23, osteocalcin, IL-6, neutrophil-to-lymphocyte ratio (NLR), von Willebrand factor (vWF), or plasminogen activator inhibitor-1 (PAI-1); and
(b) treating or continuing treatment if there is an increased level of IL-23 or osteocalcin as compared to a reference level and/or a decreased level of IL-6, NLR, vWF, or PAI-1 as compared to a reference level, and/or low tumor burden as compared to a reference level. Determining whether the biomarkers levels have increased or decreased as compared to a reference level can be determined by comparison to a reference population having similar cancers and tumors and determining the median or mean levels of expression. In particular embodiments, the level of PAI-1 is <229 pg/mL, the level of IL-6 is <5.4 pg/mL, the level of IL-23 >is 2.1 pg/mL, and the level of osteocalcin is >32 pg/mL.

In some embodiments, the method comprises treatment with durvalumab. The term “durvalumab” as used herein refers to an antibody that selectively binds PD-L1 and blocks the binding of PD-L1 to the PD-1 and CD80 receptors, as disclosed in U.S. Pat. No. 9,493,565 (wherein durvalumab is referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC). Durvalumab can relieve PD-L1-mediated suppression of human T-cell activation in vitro and inhibits tumor growth in a xenograft model via a T-cell dependent mechanism.

In some embodiments, the methods disclosed herein comprise treatment with tremelimumab. The term “tremelimumab” as used herein refers to an antibody that selectively binds a CTLA-4 polypeptide, as disclosed in U.S. Pat. No. 8,491,895 (wherein tremelimumab is referred to as “clone 11.2.1”), which is incorporated by reference herein in its entirety. Tremelimumab is specific for human CTLA-4, with no cross-reactivity to related human proteins. Tremelimumab blocks the inhibitory effect of CTLA-4, and therefore enhances T-cell activation. Tremelimumab shows minimal specific binding to Fc receptors, does not induce natural killer (NK) ADCC activity, and does not deliver inhibitory signals following plate-bound aggregation.

In some embodiments, the methods disclosed herein comprise treatment with durvalumab and tremelimumab. In some embodiments, the methods disclosed herein comprise treatment with durvalumab. In some embodiments, the methods disclosed herein comprise treatment with tremelimumab

The term “patient” is intended to include human and non-human animals, particularly mammals.

In some embodiments, the methods disclosed herein relate to treating a subject for a tumor disorder and/or a cancer disorder. In some embodiments, the cancer is head and neck cancer. In some embodiments, the head and neck cancer is a squamous cell carcinoma. In some embodiments, the cancer is recurrent and/or metastatic.

The terms “treatment” or “treat” as used herein refer to both therapeutic treatment and prophylactic or preventative measures. Those in need of treatment include subjects having cancer as well as those prone to having cancer or those in cancer is to be prevented. In some embodiments, the methods disclosed herein can be used to treat cancer. In other embodiments, those in need of treatment include subjects having a tumor as well as those prone to have a tumor or those in which a tumor is to be prevented. In certain embodiments, the methods disclosed herein can be used to treat tumors. In other embodiments, treatment of a tumor includes inhibiting tumor growth, promoting tumor reduction, or both inhibiting tumor growth and promoting tumor reduction.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, or using implants.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier” as used herein refer to one or more formulation materials suitable for accomplishing or enhancing the delivery of one or more antibodies of the disclosure.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only, and should not be construed as limiting the scope of the invention in any way.

### Example 1: Durvalumab Plus Tremelimumab or Chemotherapy Therapy for Treatment of Recurrent/Metastatic Head and Neck Squamous Cell Carcinoma

EAGLE (NCT02369874) was a randomized, open-label, phase 3 trial study that evaluated the efficacy of durvalumab (D) or durvalumab plus tremelimumab (D+T) versus chemotherapy in patients with recurrent/metastatic head and neck squamous cell carcinoma. Patients with disease progression after platinum-based CT were randomized 1:1:1 to durvalumab (10 mg/kg every 2 weeks), durvalumab plus tremelimumab (durvalumab 20 mg/kg every 4 weeks plus tremelimumab 1 mg/kg every 4 weeks for 4 doses, then durvalumab 10 mg/kg every 2 weeks), or chemotherapy (cetuximab, a taxane, methotrexate, or a fluoropyrimidine). The primary endpoint of overall survival with durvalumab versus chemotherapy, and overall survival with durvalumab plus tremelimumab versus chemotherapy was not met in the EAGLE trial; there were no statistically significant differences in overall survival with durvalumab or durvalumab plus tremelimumab versus chemotherapy. However, overall survival at landmark timepoints (12, 18, and 24 months) was higher with durvalumab than with chemotherapy, demonstrating clinical activity for durvalumab.

### Mutational Profiling and bTMB Calculation Using Plasma ctDNA

Plasma samples were profiled to identify somatic alterations including single-nucleotide variants, small indels and copy number amplifications using GuardantOMNI next-generation sequencing platform (Guardant Health, Redwood City, CA) comprising 500 genes (2.145 Mb). The OMNI TMB algorithm incorporates somatic synonymous and non-synonymous single nucleotide variants (SNVs) and short insertions/deletions (indels) at all variant allele fractions across 1.0 Mb of genomic coding sequence and is optimized to calculate TMB on plasma samples with low cell-free circulating tumor DNA content. Alterations associated with clonal hematopoiesis, germline and oncogenic driver or drug resistance mechanisms were excluded from the TMB calculation. Samples with low tumor shedding (e.g., maximum somatic allele fraction <0.3%) or low unique molecule coverage were considered bTMB-unevaluable.

### Determination of bTMB Cutoff

A series of bTMB cutoff values from 5 to 20 mut/Mb were examined to determine the optimal hazard ratio of OS for durvalumab as compared with SoC in the bTMB high cohort. Two-fold cross validation analyses were performed and a minimum p value approach based on Cox proportional hazard (PH) model was used to select the optimal cut-point from the above values. The most frequently selected cut-points in the Cox PH models in training sets were considered as potential optimal cutoffs. These potential optimal cutoffs in the training set were then validated based on HR distribution in the validation set.

### Statistical Analysis

The Kaplan-Meier method was used to calculate univariate survival estimates for progression-free survival and overall survival. Minimum p value approach based on Cox PH model, 2 folds cross validation analyses were performed. The most frequently selected minimum p value cutoffs in the Cox PH models for training sets will be consider as potential optimal cutoffs. These potential optimal cutoffs will be validated based on HR distribution from validation set. The optimal cutoff will be determined based on further exploration of the efficacy differentiation by the cutoffs using full dataset. A Cox proportional hazard model was used to define the association of mutational status of genes with PFS and OS. P-values were assessed using the log-rank test. Wilcoxon rank-sum test and Kruskal-Wallis test were used when comparing continuous variables. All p-values are two-sided. 10,000-fold cross-validation was performed to evaluate PFS and OS performance at all cutoffs evaluated. Analyses were performed using SAS and R (version 3.4.3, R Foundation, Vienna, Austria).

### Results

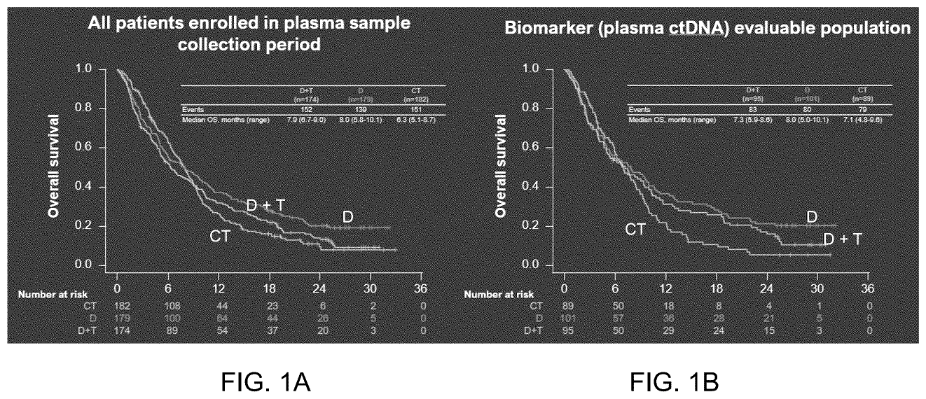

The retrospective analysis of the EAGLE trial included 736 intent-to-treat patients and 247 were evaluable for bTMB (BEP). Baseline characteristics were generally well balanced among the intention to treat population, patients enrolled in the plasma collection period, and the blood TMB evaluable populations, and were representative of a patient population with platinum-refractory recurrent/metastatic head and neck squamous cell carcinoma. When comparing all patients enrolled in the biomarker evaluable population sample collection period with the biomarker evaluable population, overall survival with durvalumab remained unchanged; however, overall survival in the chemotherapy group was higher in all samples than in the biomarker evaluable population (FIGS. 1A-1B). The differences may be due to failed samples as well as samples not collected; both factors could affect overall survival. However, the sample size was too small to make a conclusion.

TABLE 1
Baseline Characteristics of Patients in the Intention to Treat Population

| | ITT | Patients enrolled in plasma | bTMB evaluable |
|---|---|---|---|
| | (n = 736) | collection period* (n = 535) | (n = 247) |
| Age (years), median | 60 | 61 | 61 |
| Age < 65 years, n (%) | 514 (69.8) | 374 (69.9) | 173 (70.0) |
| Sex, male, n (%) | 618 (84.0) | 444 (83.0) | 208 (84.2) |
| Race, white, n (%) | 591 (80.3) | 417 (77.9) | 185 (74.9) |
| ECOG PS = 1, n (%) | 531 (72.1) | 386 (72.1) | 168 (68.0) |
| Smoking status, n (%) | | | |
| Never smoker | 155 (21.1) | 109 (20.4) | 52 (21.1) |
| Ever smoker | 581 (78.9) | 426 (79.6) | 195 (78.9) |
| Primary tumor location, n (%) | | | |
| Oral cavity | 190 (25.8) | 138 (25.8) | 57 (23.1) |
| Oropharynx | 274 (37.2) | 202 (37.8) | 100 (40.5) |
| Hypopharynx | 129 (17.5) | 98 (18.3) | 47 (19.0) |
| Larynx | 115 (15.6) | 78 (14.6) | 36 (14.6) |
| PD-L1 positive, n (%) | 212 (28.8) | 161 (30.1) | 81 (32.3) |
| HPV positive, n (%) | 141 (19.2) | 97 (18.1) | 45 (18.2) |
| Objective response, n (%) | | | |
| CR | 12 (1.6) | 10 (1.9) | 6 (2.4) |
| PR | 119 (16.2) | 89 (16.6) | 42 (17.0) |
| SD | 204 (27.7) | 137 (25.6) | 65 (26.3) |
| PD | 355 (48.2) | 267 (49.9) | 125 (50.6) |

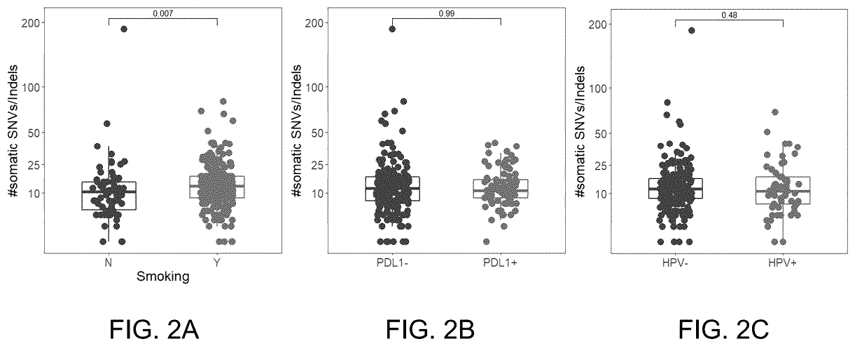

Guardant OMNI panel was applied to 300 plasma samples from baseline, and data were successfully generated for 286 (95%) patients. Somatic SNVs or indels were detected in 279 (98%) patients and the median variant count per sample is 12 (FIGS. 2A-2C). Patients with smoking history showed significantly higher number of somatic SNVs or indels than patients who were never smokers (median number 13 versus 10.5, P=0.007, Wilcoxon rank-sum test), which is consistent with the understanding that carcinogens in tobacco could cause DNA damages and thus gene mutations (FIG. 2A). However, no association was observed between somatic mutation counts and PD-L1 or HPV status, similar to previous report in treatment naïve patients (TCGA).

Somatic mutations were identified in 387 genes and were found in more than 20% of samples in 7 genes, including TP53 (79%), KMT2D (33%), FAT1 (26%), LRP1B (23%), TERT (23%), PIK3CA (22%) and NOTCHI (21%). The prevalence of TP53 mutations is comparable with 72% reported by TCGA, and a higher prevalence (86%) was found in HPV-ve patients, consistent with previous observation (Leemans et al., Nat. Rev. Cancer 18(5): 269-82 (2018)). The prevalence of FAT1, LRP1B, PIK3CA, and NOTCH1 mutations is also similar to that in TCGA cohort (23%, 20%, 21% and 19%, respectively), suggesting the somatic mutational landscape in R/M HNSCC is generally consistent with treatment naïve HNSCC. Notably in the cohort, KMT2D gene showed increased mutation frequency as compared with TCGA cohort (33% versus 18%), which may imply more prevalent epigenetic rewiring in R/M HNSCC. 59 TERT promoter mutations in 57 patients were also reported, including two recurrent mutations (−124 G>A, N=34 and −146 G>A, N=11). Expression of TERT tend to be enhanced by bearing those promoter mutations and could promote unlimited cell growth (Shay et al., Semin. Cancer Biol. 21(6): 349-53 (2011)), highlighting its critical role in HNSCC carcinogenesis. High mutation frequencies of several homologous recombination DNA damage repair genes (Heeke et al., JCO Precis. Oncol. (2018), doi: 10.1200/P0.17.00286) were observed in the R/M HNSCC cohort, including ATM (15%), CHEK2 (12%), and ARID1A (11%), which are significantly elevated in contrast to treatment naïve cohort (3%, 2%, and 4%, respectively).

Since it is challenging to identify copy number loss in plasma circulating free DNA (cfDNA), only amplifications were reported in this study. In total, 878 amplifications were identified in 98 genes and 145 patients. For patients with amplifications, a median of three were found. Consistent with TCGA cohort, cyclin D1 (CCND1) on 11q13 was the most frequently observed amplification, presented in 25% of patients. HPV−ve tumors were more prone to CCND1 amplification as compared with HPV+ve tumors (29% versus 10%, P=0.0034, Fisher's test), indicating potential different mechanisms in tumor development. The other genes with recurrent amplifications in more than 10% of the cohort include FGF3 (25%), FGF19 (19%), PIK3CA (18%), and PIK3CB (17%), in general concordance with previous reports. Notably, CCND1, FGF3, and FGF19 were all on 11q13 and they were co-amplified in most of patients.

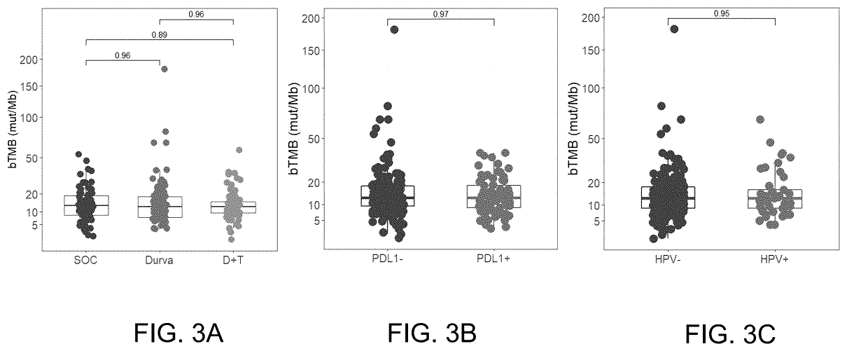

bTMB data from 247 patients enrolled in EAGLE was generated. The median bTMB of EAGLE cohort was 12.6 (mut/Mb). 74 (30%) or 50 (20%) patients showed bTMB ≥16 or ≥20, respectively. The bTMB distribution across all three arms was similar (FIGS. 3A-3C), and was independent of PDL1 and HPV status.

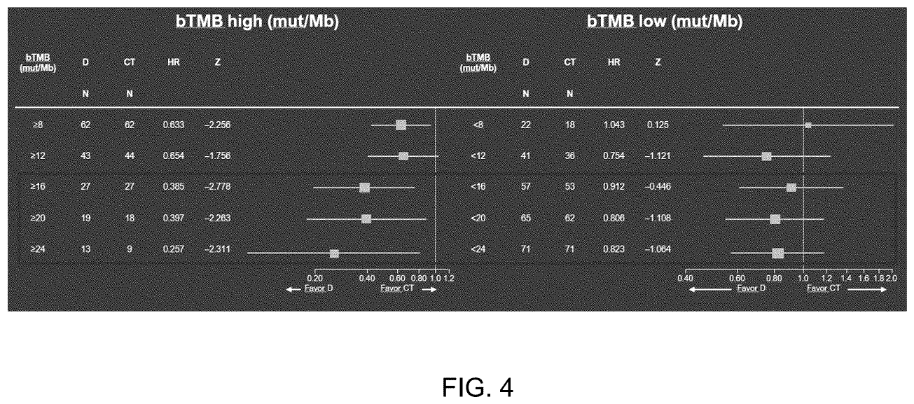

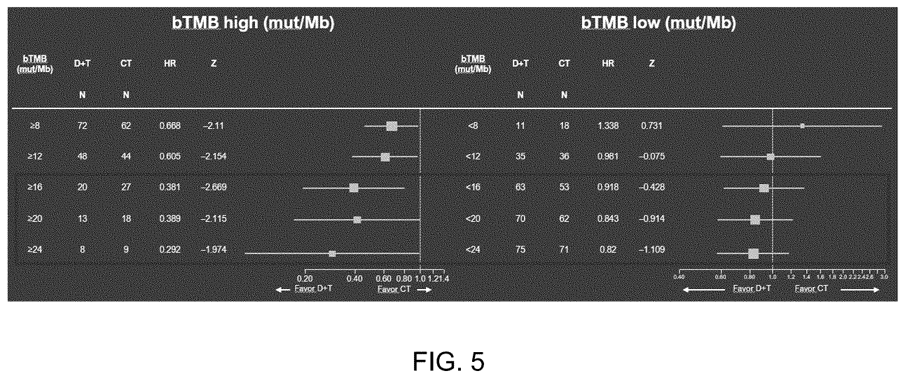

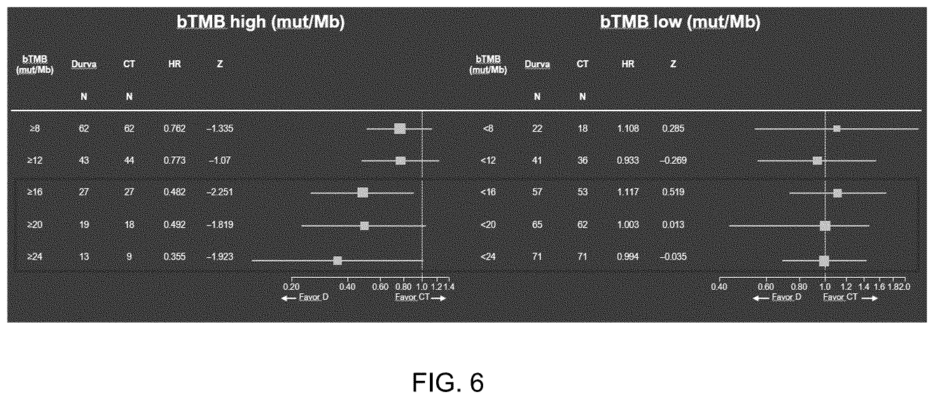

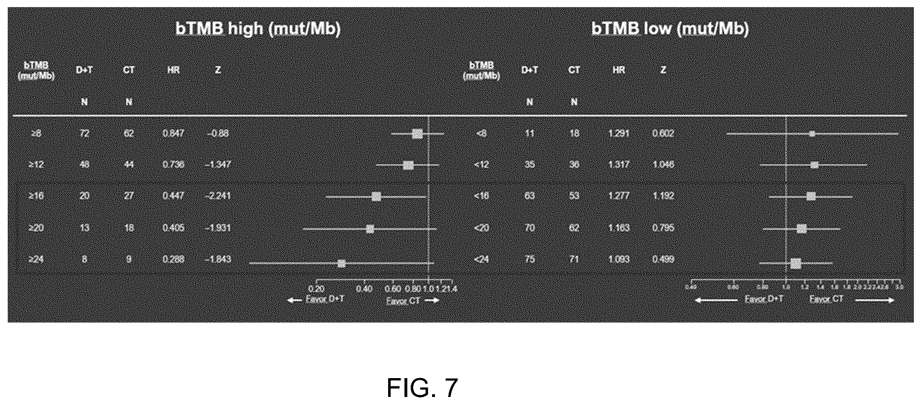

Patients were stratified into bTMB high and bTMB low subgroups using different cutoffs. In the bTMB high cohort, a clear signal of significantly improved overall survival was found in both durvalumab and durvalumab plus tremelimumab treatment arms as compared with SoC arm, when using bTMB cutpoints greater than or equal to 16 mutations per megabase (FIGS. 4 and 5). The benefit of durvalumab and durvalumab plus tremelimumab versus SoC in patients with high bTMB generally improved with increasing cutoff. However, no such benefit of durvalumab and durvalumab plus tremelimumab could be observed for bTMB low patients. The same pattern was also found for PFS (FIGS. 6 and 7), highlighting that in R/M HNSCC, bTMB is a predictive biomarker for durvalumab and durvalumab plus tremelimumab treatments, which can significantly improve OS and PFS in patients with high bTMB.

TABLE 2
Overall Hazards for High versus low bTMB

| bTMB | | | | |
|---|---|---|---|---|
| cutoff | D vs CT | | D + T vs CT | |
| (mut/Mb) | High | Low | High | Low |
| ≥8 | 0.63 (0.42-0.94) | 1.04 (0.54-2.03) | 0.68 (0.46-0.98) | 1.34 (0.61-2.92) |
| ≥12 | 0.65 (0.41-1.05) | 0.75 (0.46-1.23) | 0.61 (0.39-0.97) | 0.98 (0.60-1.61) |
| ≥16 | 0.39 (0.20-0.75) | 0.91 (0.61-1.37) | 0.40 (0.20-0.81) | 0.92 (0.62-1.36) |
| ≥20 | 0.40 (0.18-0.88) | 0.81 (0.55-1.18) | 0.41 (0.17-1.00) | 0.84 (0.58-1.22) |
| >24 | 0.26 (0.08-0.81) | 0.82 (0.57-1.18) | 0.29 (0.09-0.99) | 0.83 (0.58-1.17) |

Cross-validation also supported 16 mutations per megabase was the optimal bTMB cut-point in the EAGLE study. When incorporating this cut-point to stratify patients, no link was found between bTMB level and human papillomavirus status, PD-L1 status, age, or gender. Smoking and progression within 6 months on multi-modality chemotherapy in localized disease trended with higher bTMB. Other parameters with a greater than 5% difference between bTMB high and low subgroups include primary tumor location or Eastern Cooperative Oncology Group (ECOG) performance status and complete response rate.

TABLE 3
Baseline Characteristics of Patients based on bTMB Stratification

| | bTMB ≥ 16 | bTMB < 16 | bTMB evaluable |
|---|---|---|---|
| | (n = 74) | (n = 173) | (n = 247) |
| Age (years), median | 60 | 61 | 61 |
| Age < 65 years, n (%) | 54 (73.0) | 119 (68.4) | 173 (70.0) |
| Sex, male, n (%) | 64 (86.5) | 144 (83.2) | 208 (84.2) |
| Race, white, n (%) | 56 (75.7) | 129 (74.6) | 185 (74.9) |
| ECOG PS = 1, n (%) | 53 (71.6) | 115 (66.5) | 168 (68.0) |
| Smoking status, n (%) | | | |
| Never smoker | 12 (16.2) | 40 (23.1) | 52 (21.1) |
| Ever smoker | 62 (83.8) | 133 (76.9) | 195 (78.9) |
| Primary tumor | | | |
| location, n (%) | | | |
| Oral cavity | 16 (21.6) | 41 (23.7) | 57 (23.1) |
| Oropharynx | 26 (35.1) | 74 (42.8) | 100 (40.5) |
| Hypopharynx | 18 (24.3) | 29 (16.8) | 47 (19.0) |
| Larynx | 12 (16.2) | 24 (13.9) | 36 (14.6) |
| PD-L1 positive, n (%) | 23 (31.1) | 58 (33.5) | 81 (32.3) |
| HPV positive, n (%) | 12 (16.2) | 33 (19.1) | 45 (18.2) |
| Objective response, n (%) | | | |
| CR | 5 (6.8) | 1 (0.6) | 6 (2.4) |
| PR | 14 (18.9) | 28 (16.2) | 42 (17.0) |
| SD | 18 (24.3) | 47 (27.2) | 65 (26.3) |
| PD | 35 (47.3) | 90 (52.0) | 125 (50.6) |

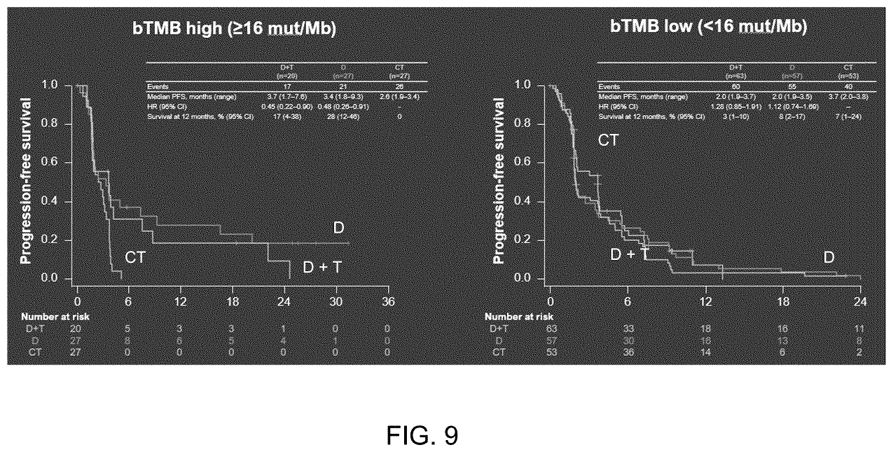

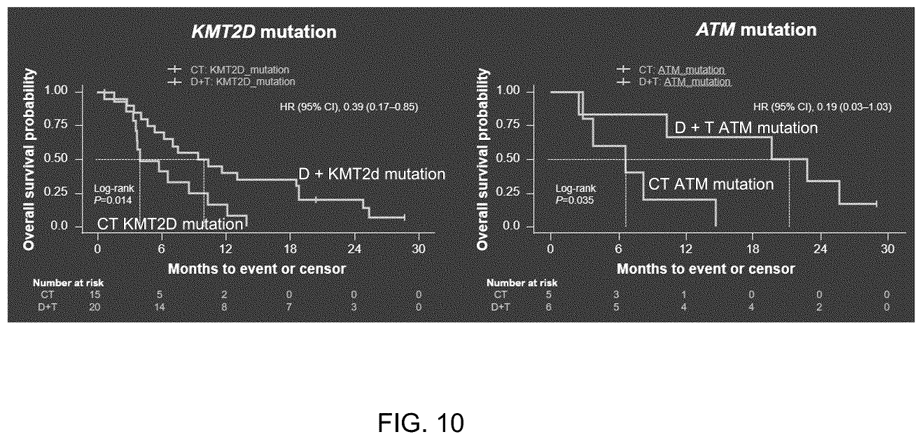

Overall survival and progression free survival in EAGLE was improved in bTMB high subgroup for durvalumab and durvalumab plus tremelimumab (FIGS. 9 and 10). The 18-month overall survival rates were 22 percent higher for durvalumab plus tremelimumab and 33 percent for durvalumab versus chemotherapy in patients with high bTMB. The 12-month overall survival rates were 17% higher for durvalumab plus tremelimumab and 28% for durvalumab versus chemotherapy in patients with high blood TMB.

Patients with pathogenic or likely pathogenic mutations in KMT2D, a head and neck squamous cell carcinoma tumor suppressor gene, showed improved overall survival for durvalumab plus tremelimumab versus chemotherapy, with a hazard ratio of 0.39 and a 95% confidence interval of 0.17 to 0.85. A trend of improved overall survival for durvalumab plus tremelimumab versus chemotherapy was also seen in patients with ATM mutations.

### Example 2: Determination of PD-L1 Assay Scoring Algorithm in Head and Neck Cancer Patients

With the availability of efficacy data from HAWK and CONDOR studies (Zandberg et al., Eur. J Cancer. 107: 142-52 (2019); Siu et al., JAMA Oncol. 5(2): 195-203 (2019)), it has been possible to analyze larger data sets, and use overall survival data to derive a PD-L1 diagnostic algorithm which is more predictive of OS. This Example illustrates the analysis used to determine an optimal algorithm for HNSCC, and the methodology used to score PD-L1 in tumors of patients. The optimal algorithm was determined as ≥50% of tumor cell or ≥25% of tumor-associated immune cells (TC≥50 or IC≥25) membrane staining for PD-L1 at any intensity, as assessed by the VENTANA PD-L1 (SP263) Assay.

Data was used from D4193C00001 (HAWK) and D4193C00003 (CONDOR) Phase II studies, in 2nd line R/M HNSCC patients. Both studies required PD-L1 status as enrollment criteria, and at screening, patient's tumor specimens were stained and scored with the VENTANA PD-L1 (SP263) Assay. Tumor cell PD-L1 expression data was available in the following bins: <1, 1-4, 5-9, 10-19, 20-24 (CONDOR), 25, 30 (26-34), 40 (35-44), 50 (45-54), 60 (55-64), 70 (65-74), 75, 80 (76-84), 90 (85-94), and 100 (95-100) (HAWK). Exploratory data was collected for immune cells, using a raw score for immune cell positivity.

Overall survival data for the patients treated with monotherapy durvalumab in these two studies was pooled. Data from the durvalumab +tremelilumab combination was not used, because there was no data from patients with PD-L1 TC≥25%. The pooled monotherapy data was from a total of 179 subjects (112 subjects from the HAWK study and 67 subjects from the CONDOR study). The PD-L1 prevalence in the pooled monotherapy group was 62%, whereas the prevalence in a natural population is 25-30%.

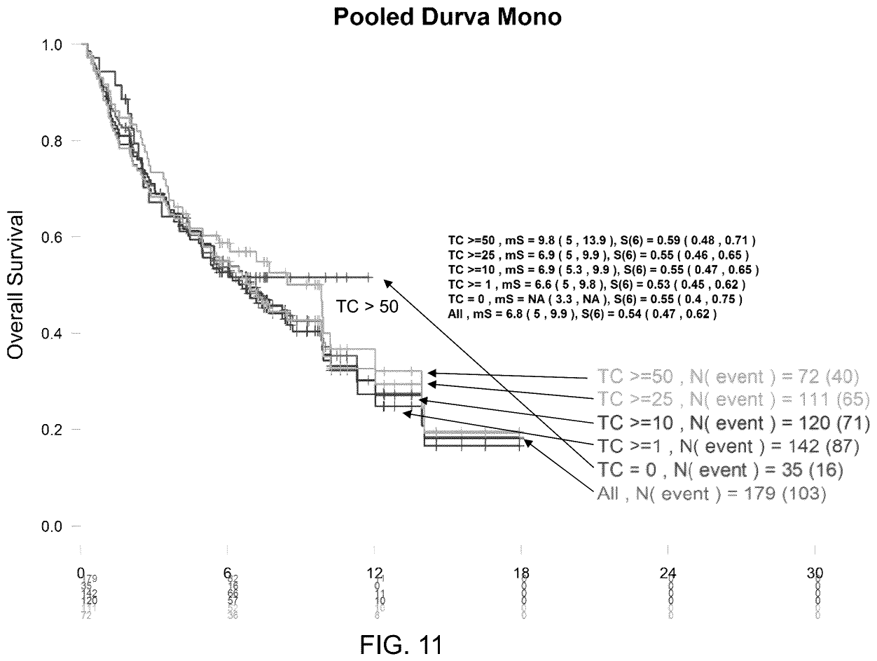

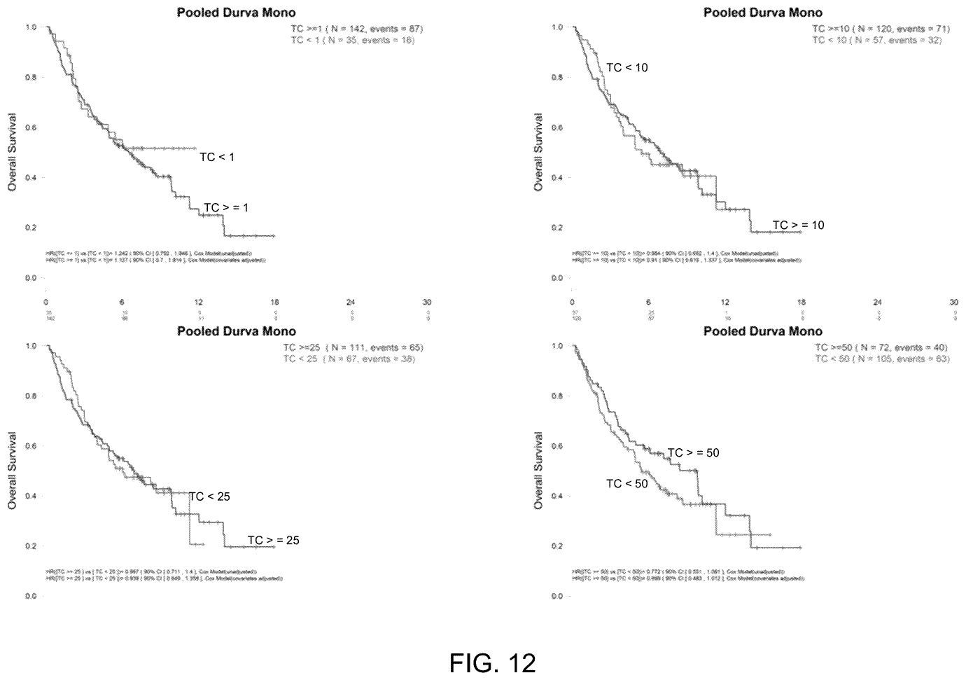

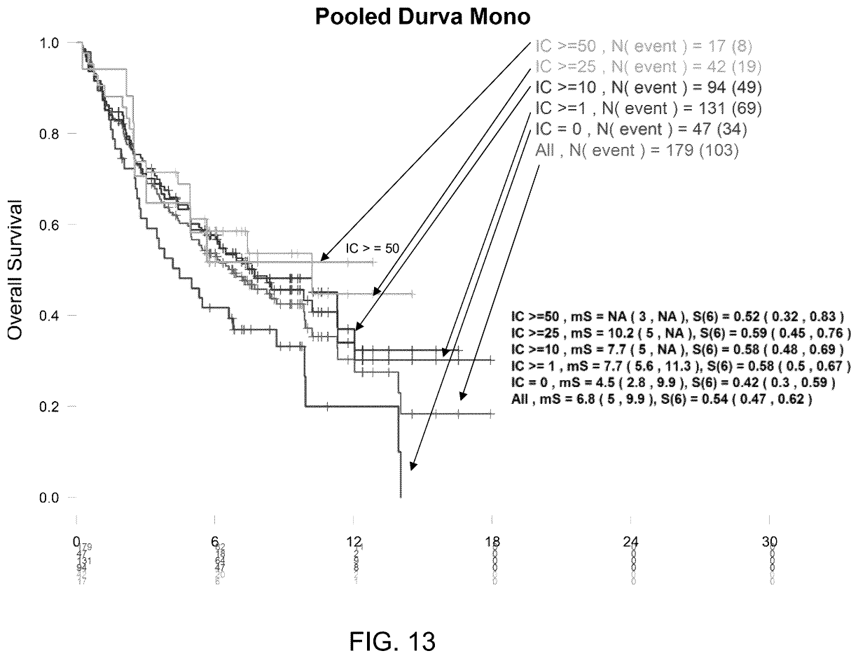

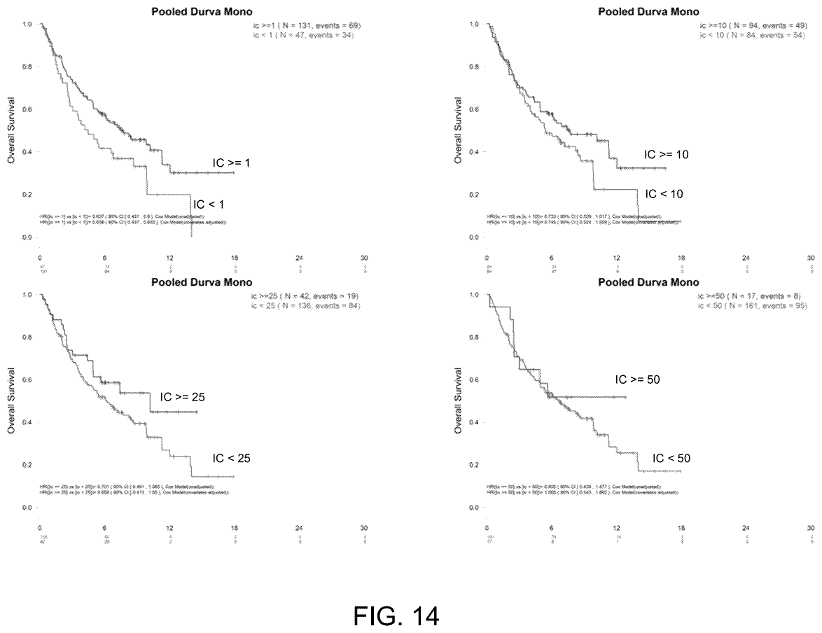

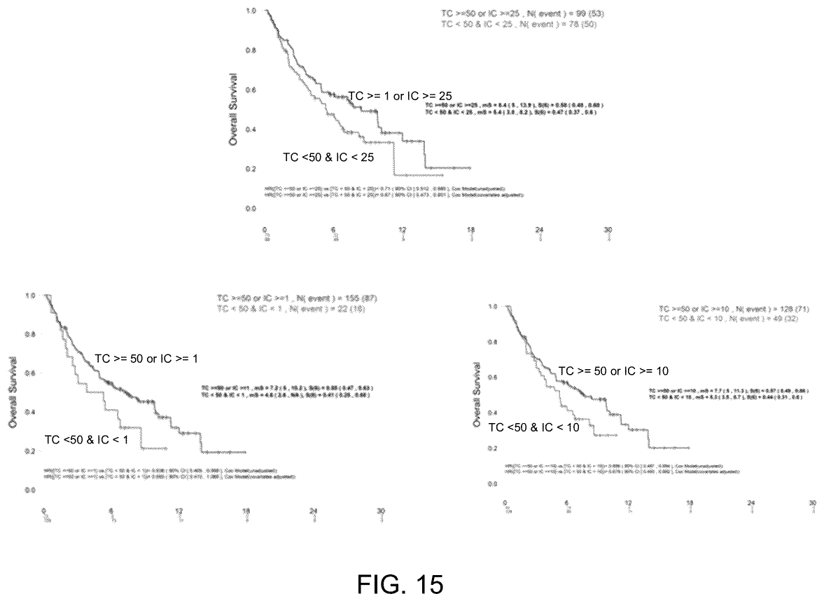

There was a general trend of increasing survival (median and 6 month) with increasing Tumor Cell PD-L1 expression (FIG. 11). Overall survival was determined for patients with 0%, >1%, >10% >25%, >50% Tumor cell PD-L1 expression. The highest median survival was seen for patients with TC>=50% PD-L1 expression. The cut-off of TC50% best discriminated a subgroup of patients with better survival (TC>=50%) from a PD-L1 low subgroup (TC<50%) (FIG. 12). Based on this data TC>=50% was selected as the tumor cell cut-off. The data showed a trend of increasing median survival, with increasing immune cell expression, except at IC>=50%. (FIG. 13). Based on this data, it was decided to include immune cell positivity in the scoring algorithm. At cut-off of IC1, 10, and 25% there was good separation of patients with PD-L1 high and low expression (FIG. 14). Therefore, these were all considered as suitable for combination with the TC50% cut-off. For all algorithms the TC/IC PD-L1 high subgroups showed superior median survival to the corresponding PD-L1 low subgroup. Of these, the highest median overall survival was seen with TC≥50 or IC≥25% (FIG. 15).

The algorithm TC>=50% or IC>=25% was considered most technically feasible. Based on experiences with the Urothelial Cancer SP263 (Zajac et al., 2016, European Society Medical Oncology (ESMO) Poster 26P), IC≥25% was considered likely to be more reproducible (higher intra-reader precision) than IC10 or IC1, and thus would make a more robust diagnostic assay in the clinic.

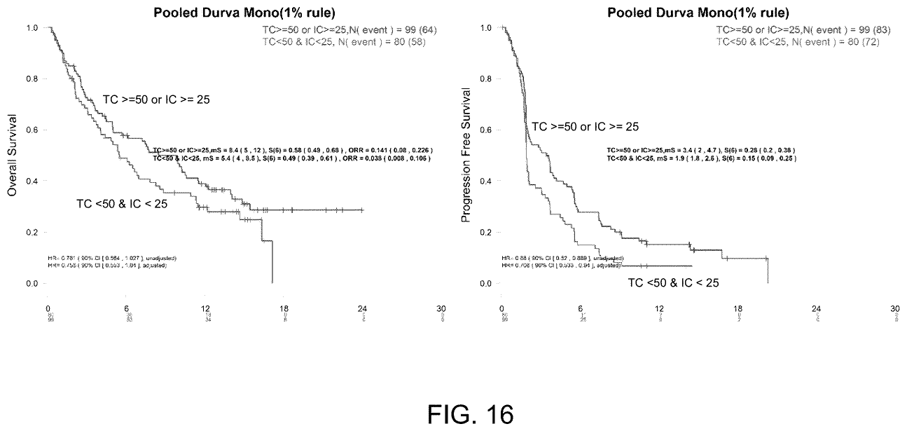

A later analysis of more mature data was used to confirm the cut-off. Data maturity was 68% for OS and 85% for PFS. The HR was 0.758 (adjusted). PFS was 3.4 months v 1.9 months in PD-L1 high v PD-L1 low (FIG. 16).

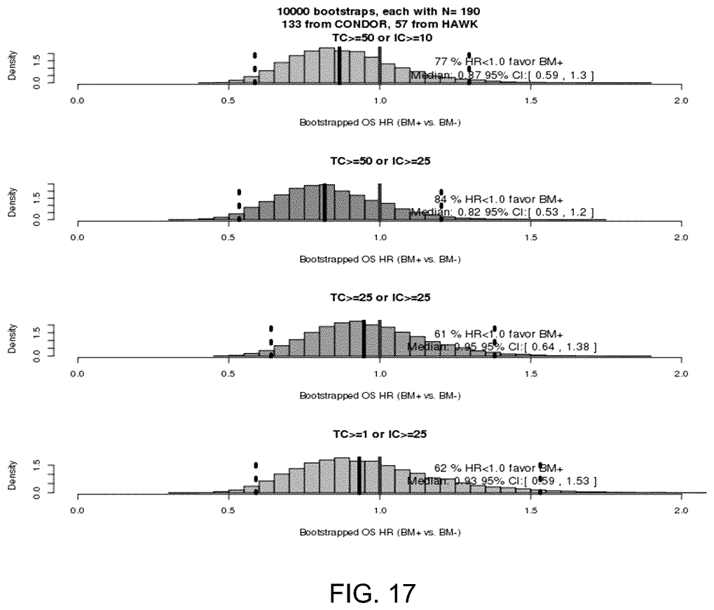

Pooled data from the HAWK/CONDOR studies did not represent a natural prevalence. In order to model the all-comers population a boot-strapping OS hazard ratio (HR) analysis was performed across the various TC/IC subgroups. Data showed the cut-point of TC≥50 or IC≥25% was optimal with the lowest HR (FIG. 17).

In order to classify HNSCC patients based on the PD-L1 TC/IC scoring algorithm, PD-L1 expression in tumor cell (TC) and tumor-associated immune cells (IC) was detected by VENTANA PD-L1 (SP263) Assay in formalin-fixed, paraffin-embedded (FFPE) head and neck squamous cell carcinoma (HNSCC). An isotype matched negative control antibody was used to evaluate the presence of background in test samples and establish a baseline staining intensity.

PD-L1 status and expression was assigned by a trained pathologist based on their evaluation of the percentage of specific staining for both tumor and tumor-associated immune cells (macrophages, dendritic cells, and lymphocytes). PD-L1 status was determined by the percentage of tumor cells with any membrane PD-L1 staining above background or by the percentage of tumor-associated immune cells with PD-L1 staining at any intensity above background.

Immune cell scoring was performed by first calculating the percentage of immune cells present as a proportion of the tumor environment (ICP-value) on the H&E section. The ICP value was expressed in individual percentages. The IC-score was generated by expressing the percentage of positive PD-L1 immune cells as a proportion of the ICP-value. PD-L1 high expression level was greater than or equal to 50% tumor cells with PD-L1 membrane staining or greater than or equal to 25% immune cell PD-L1 staining. PD-L1 low was defined as both <50% TC and <25% IC with membrane staining for PD-L1 at any intensity (Table 4).

In cases where the ICP was equal to 1%, IC positivity (IC+) was scored as either 0%, <100%, or 100% due to the difficulties in estimating the percent staining in small volumes of immune cells in low measures. The small amount of PD-L1 staining observed in cases with <100% IC positivity, should be considered as <25% PD-L1 expression.

TABLE 4
Patient classification based on PD-L1 expression in
the Ventana interpretation guide follows the algorithm below:

| | TC ≥ 50% | TC < 50% |
|---|---|---|
| IC ≥ 25% | PD-L1 High | PD-L1 High |
| IC < 25% | PD-L1 High | PD-L1 Low |

### Example 3: TMB and Other Biomarkers for their Predictive Potential in Patients Treated with Durvalumab (D) or Durvalumab +Tremelimumab (D+T) in HAWK and CONDOR Trials

A retrospective analysis was performed to evaluate TMB and other biomarkers for their predictive potential in patients benefiting from durvalumab (D) or durvalumab +tremelimumab (D+T) in 2 trials of R/M HNSCC. In the single-arm, Phase II HAWK study (Zandberg et al., Eur. J. Cancer. 107: 142-52 (2019)), 112 patients (PD-L1 tumor cell [TC] staining ≥25%) received D (10 mg/kg every 2 weeks [Q2W] for ≤12 months [mo]). In the randomized, open-label, Phase II CONDOR trial (Siu et al., JAMA Oncol. 5(2): 195-203 (2019)), 67 patients (PD-L1TC<25%) received D (10 mg/kg Q2W for ≤12 mo), 133 received D+T (D 20 mg/kg every 4 weeks [Q4W], T 1 mg/kg Q4W for ≤12 mo), and 67 received T (10 mg/kg Q4W for 7 doses then Q12W for 2 additional doses for ≤12 mo). Interactions of PD-L1 and TMB as predictive biomarkers were also evaluated.

Paired formalin-fixed, paraffin-embedded (FFPE) archival tumor and peripheral blood mononuclear cell (PBMC) samples (as germline controls) in the HAWK and CONDOR trials were evaluated by whole exome sequencing (WES). HLA class 1 types were obtained using WES of PBMC. Human papillomavirus (HPV) was assessed locally using any WES method or centrally using p16 immunohistochemistry. Neutrophil-to-lymphocyte ratio (NLR) was assessed locally. Statistical analyses included the Wilcoxon test, log-rank test, and Cox proportional hazards model. PD-L1 expression status was determined using the VENTANA PD-L1 (SP263) Assay and a cutoff of TC≥25%.

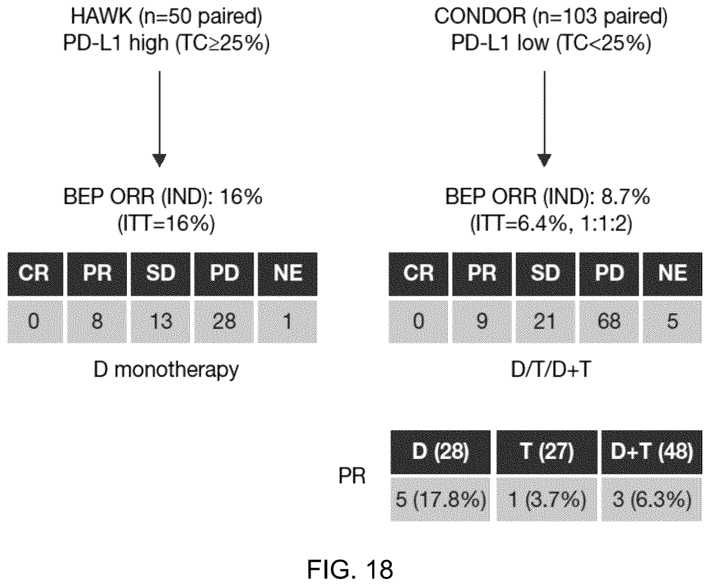

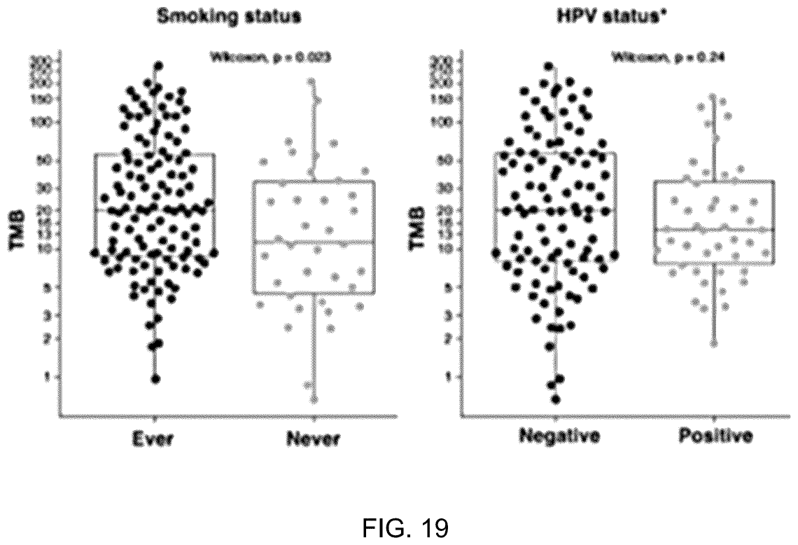

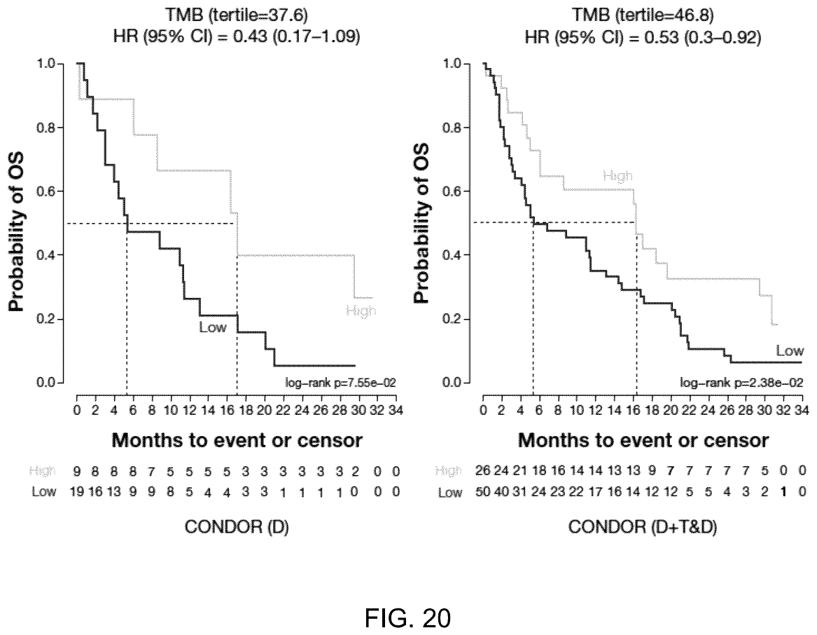

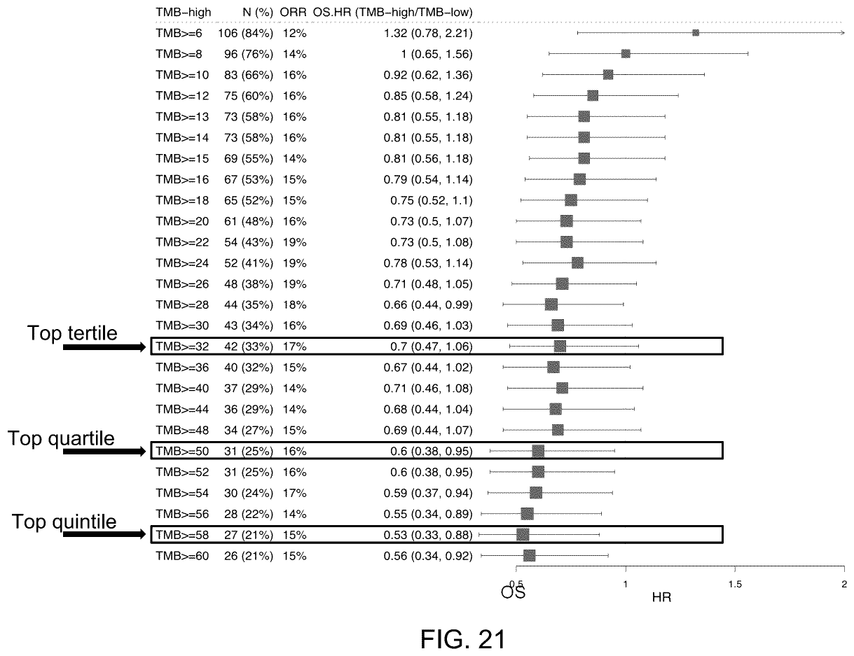

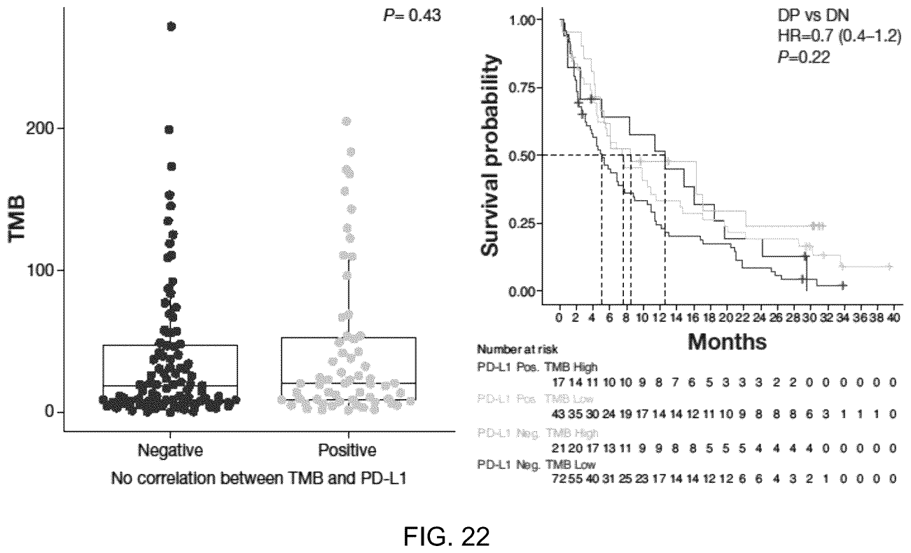

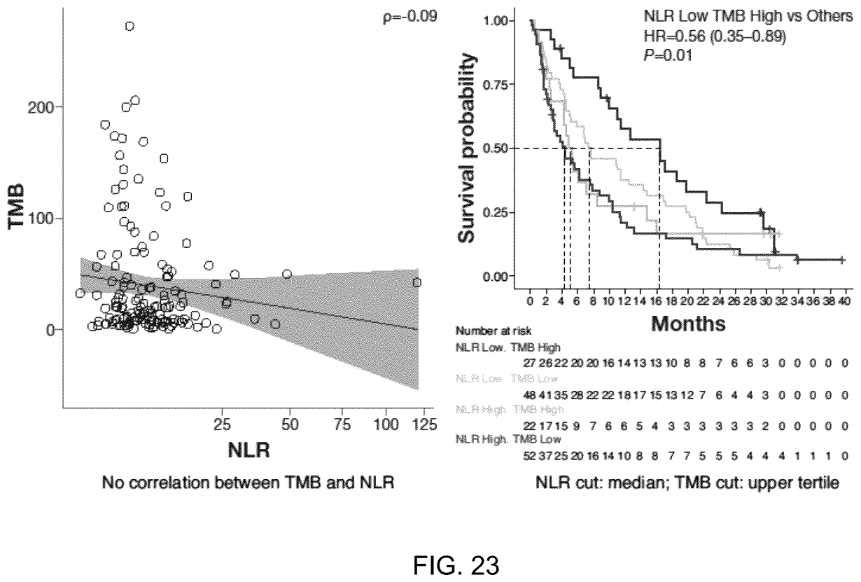

In the HAWK and CONDOR trials, 153 patients had evaluable FFPE samples (FIG. 18). TMB distributions were comparable between studies. TMB correlated with smoking (P=0.02) but not with HPV status (P=0.24) (FIG. 19). TMB also did not correlate with PD-L1 status. In the CONDOR study, high TMB (≥upper tertile) was associated with longer overall survival (OS) as compared with low TMB (FIG. 20). For combined D and D+T (N=76), OS was significantly longer with high versus low TMB (16.3 vs 5.3 mo; hazard ratio [HR]=0.53; 95% confidence interval [CI], 0.30-0.92; P=0.0238). TMB and OS association was further assessed by increasing TMB cutoffs (FIG. 21). Improved HRs trended with higher cutoffs. Cutoffs ≥upper quartile were significantly linked to OS. In combined HAWK/CONDOR analysis of patients with double negative PD-L1 and TMB (FIG. 22), patients with low PD-L1 and low TMB had the shortest OS as compared to those with high PD-L1 or high TMB. Patients with low NLR (<median) and high TMB (≥upper tertile) had significantly better OS than other patients. In patients with high NLR (≥median), TMB status did not appear to impact OS (FIG. 23). Analysis of germline HLA alleles revealed poorer survival for carriers of the HLA-B*15:01 allele (9.4%) (HR=1.91; 95% CI, 1.22-2.97; P=0.004). There was a trend toward longer OS in carriers of the HLA-B*44 allele (31.8%) as compared with non-carriers (HR=0.77; 95% CI, 0.57-1.03; P=0.08). Germline HLA heterozygosity was not a predictor of OS in patients from HAWK and CONDOR (79.2% were HLA heterozygous) (HR=1.09; 95% CI, 0.79-1.51; P=0.59).

### Example 4: Overall Survival Modeling and Association with Serum Biomarkers in Durvalumab Treated Patients with Head and Neck Cancer

Pooled longitudinal tumor size, survival, and dropout data from 4 trials involving 467 patients with HNSCC were used to develop tumor size-driven hazard models (1108: NCT01693562, CONDOR: NCT02319044, HAWK: NCT02207530, and EAGLE: NCT02369874). A Tumor Growth Inhibition (TGI) Model was developed using non-linear mixed effects methods to characterize the longitudinal tumor size data. The model primarily assumed that the total tumor volume (Tₜₒₜₐₗ) included sensitive (Tₛₑₙₛ) and insensitive (Tᵢₙₛₑₙₛ) tumor compartments to anti-programmed death ligand-1 treatment. A capacity-limited logistic growth function was used to model growth in the insensitive compartment whereas the sensitive compartment was modeled with first order growth (kg) and second order shrinkage rate (kkkill).

T′ₛₑₙₛ=[(kg×Tₛₑₙₛ)]−kₖᵢₗₗ×T²ₛₑₙₛ×Rᵢₘ(t)

Tᵢₙₛₑₙₛ=[(kg×Tᵢₙₛₑₙₛ)]×[(1−Tᵢₙₛₑₙₛ/Tₘₐₓ)])

where Rim(t) denotes a delay function constrained between 0 and 1 via transduction through transit compartments where maximum tumor shrinkage effect occurs at Rim(t)=1. The fraction of sensitive tumor cells at baseline is estimated as Fₛₑₙₛ (=Tₛₑₙₛ(0)/Tₜₒₜₐₗ(0)). The mean transit time for the delay function (D_{TIM}) was characterized using a mixture model, with 2 distinct populations:
Population 1: no delay (D_{TIM}=0)
Population 2: log-normally distributed around a non-zero value (D_{TIM}>0).
   Overall Survival (OS) and study dropout were modeled using the following relationships:

h_OS (t)=HZₒₛ×exp(0_{TS})×TS (t)

h_DO (t)=HZ_{do}×α×t^{α}×exp(0_{PCT_TS})×PCT_TS (t)×exp(0_{TBSL})×TBSL,

where h_OS(t) and h_DO(t) are hazard of death and dropout at time t, respectively; HZₒₛ and HZ_{do} respectively denote the baseline hazard for death and dropout; α is the shape parameter of the Weibull function; TS (t), and PCT_TS (t) are model-predicted tumor size and change from baseline tumor size, at time t for each individual, respectively. TBSL is the tumor size at baseline.

Covariate analyses were performed on the TGI, OS, and dropout models. The full model approach covariate modeling followed by univariate backward elimination (based on a type-I error of 5%) was used to identify significant biomarkers. A panel of 66 serum protein biomarkers at baseline and 4 relevant clinical markers from 346 out of 413 patients treated with durvalumab (all studies except 1108) were initially screened to select a pool of 21 candidate covariates. The criteria for dimensionality reduction comprised correlation strength between biomarkers and pharmacological hypotheses pertaining to a prior analysis (inflammation, immunomodulation, tumor burden, and angiogenesis).

Cut-point and regression analysis using the final baseline predictors of survival to identify subsets of patients with substantial survival benefits were used. Similar baseline tumor burden and most inflammatory markers were observed across the legacy studies (Table 5). Of note, cross study effects were observed for some of the measured serum cytokines (data not shown), which were assessed and accounted for during the multivariate analysis.

TABLE 5
Baseline Covariate Distribution Across Studies

| CONDOR (N = 49) | | | EAGLE (N = 209) | | HAWK (N = 88) | | P value |
|---|---|---|---|---|---|---|---|
| IL-6 | | | | | | | 0.171 |
| Mean (SD) | 7.9 | (7.6) | 10.0 | (11.2) | 7.7 | (12.0) | |
| Range | 4.1-45.0 | | 5.4-116.0 | | 4.1-106.0 | | |
| IL-23 | | | | | | | <0.001 |
| Mean (SD) | 1.8 | (0.4) | 2.2 | (0.5) | 1.7 | (0.3) | |
| Range | 1.5-2.9 | | 1.8-4.7 | | 1.5-3.0 | | |
| Osteocalcin | | | | | | | 0.048 |
| Mean (SD) | 67.8 | (46.8) | 55.4 | (38.0) | 66.1 | (46.0) | |
| Range | 8.5-245.0 | | 5.4-221.0 | | 2.7-263.0 | | |
| PAI-1 | | | | | | | 0.002 |
| Mean (SD) | 262.3 | (116.8) | 230.6 | (79.9) | 269.7 | (110.4) | |
| Range | 44.0-737.0 | | 80.0-504.0 | | 111.0-719.0 | | |
| VEGF | | | | | | | 0.349 |
| Mean (SD) | 467.9 | (433.0) | 415.1 | (246.8) | 394.3 | (270.8) | |
| Range | 63.0-2320.0 | | 44.0-1240.0 | | 44.0-1460.0 | | |
| vWF | | | | | | | 0.519 |

| | | | | | | |
|---|---|---|---|---|---|---|
| Mean (SD) | 232.9 | (110.2) | 386.0 | (1541.9) | 235.2 | (109.3) |

| | | | |
|---|---|---|---|
| Range | 49.0-548.0 | 86.0-22500.0 | 76.0-554.0 |

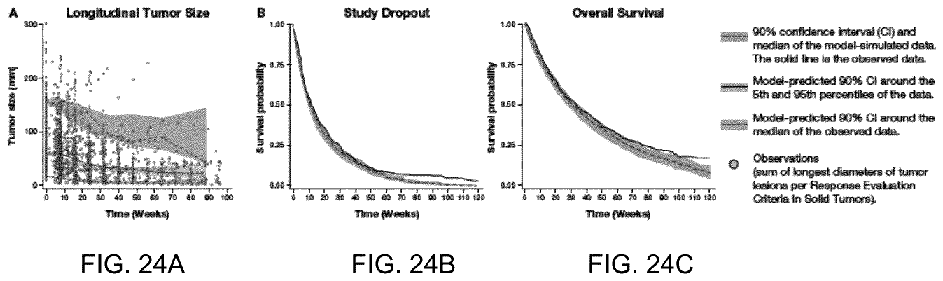

The final tumor size model highlighted that high tumor burden was associated with faster tumor growth while patients with lower baseline tumor burden had an increase in net tumor shrinkage (FIGS. 24A-24C). A favorable biomarker profile was identified by cut-point analysis using a univariate approach and combining the final results.

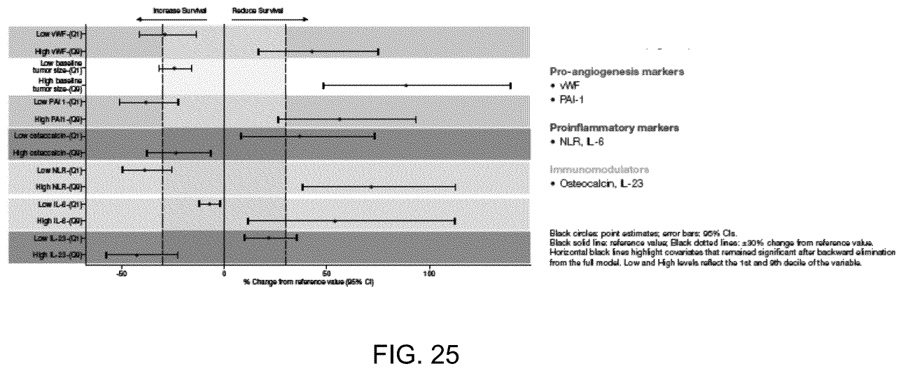

Patients with a favorable biomarker profile had high baseline levels of immunomodulators (IL-23, osteocalcin), low systemic inflammation (IL-6, NLR), low tumor burden, and low angiogenesis factors (vWF, plasminogen activator inhibitor-1 (PAI-1)) were associated with survival benefit for patients with HNSCC treated with durvalumab. Specifically, patients with a favorable biomarker profile had a combination of baseline levels of low serum PAI-1<229 pg/mL, low serum IL-6<5.4 pg/mL, high serum IL-23>2.1 pg/mL and/or high osteocalcin>32 pg/MI (FIG. 25). The serum biomarker profile of HNSCC patients with median survival times exceeding 1 year can advantageously be used for patient enrichment. The final tumor size model highlighted that high tumor burden, and elevated LDH and NLR were associated with faster tumor growth while patients with lower baseline tumor burden had an increase in net tumor shrinkage.

The tumor size model covariate analysis results revealed that as compared to the median, patients with elevated (90th percentile) serum LDH and NLR had on average 40% faster tumor growth.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present invention.

1. A method of predicting success of head and neck cancer treatment in a patient in need thereof, comprising determining the patient's tumor mutational burden (TMB), wherein a high TMB predicts success of treatment.

2. The method of claim 1, wherein a high TMB is defined as ≥16 mutations/megabase (mut/Mb).

3. The method of claim 1, wherein a high TMB is defined as ≥20 mutations/megabase (mut/Mb).

4. The method of claim 1,
wherein the treatment comprises:
(a) treatment with durvalumab;
(b) treatment tremelimumab; or
(c) treatment with both durvalumab and tremelimumab.

5. (canceled)

6. The method of claim 1, wherein the head and neck cancer is a squamous cell carcinoma.

7. The method of claim 1, wherein the head and neck cancer is a recurrent cancer or a metastatic cancer.

8. (canceled)

9. The method of claim 1, wherein the patient has a smaller neutrophil-to-lymphocyte ratio as compared to a reference level.

10. The method of claim 1, wherein ≤25% of the patient's tumor-associated immune cells express PD-L1 and/or ≤50% of the patient's tumor cells express PD-L1.

11. A method of treating head and neck cancer in a patient in need thereof, comprising:
(a) determining the patient's TMB;
(b) determining whether the TMB is high or low; and
(c) treating or continuing treatment if TMB is high or not treating or discontinuing treatment if TMB is low.

12. The method of claim 11, wherein a high TMB is defined as ≥16 mutations/megabase (mut/Mb).

13. The method of claim 11 or claim 12, wherein a high TMB is defined as ≥20 mutations/megabase (mut/Mb).

14. The method of claim 11, wherein the treatment comprises:
(a) treatment with durvalumab;
(b) treatment tremelimumab; or
(c) treatment with both durvalumab and tremelimumab.

15. (canceled)

16. The method of claim 11, wherein the head and neck cancer is a squamous cell carcinoma.

17. The method claim 11, wherein the head and neck cancer is recurrent or a metastatic cancer.

18. (canceled)

19. The method of claim 11, wherein the patient has a smaller neutrophil-to-lymphocyte ratio as compared to a reference level.

20. The method of claim 11, wherein ≤25% of the patient's tumor-associated immune cells express PD-L1 and ≤50% of the patient's tumor cells express PD-L1.

21. The method of claim 11, wherein success of treatment is determined by an increase in overall survival as compared to standard of care.

22. The method of claim 11, wherein success of treatment is determined by an increase in progression free survival as compared to standard of care.

23. A method of treating head and neck cancer in a patient in need thereof, comprising:
(a) determining whether the patient has a somatic mutation in at least one of Lysine Methyltransferase 2D (KMT2D) gene or Ataxia-Telangiectasia Mutated (ATM) gene; and
(b) treating or continuing treatment if the patient has a somatic mutation in at least one of Lysine Methyltransferase 2D (KMT2D) gene or Ataxia-Telangiectasia Mutated (ATM) gene.

24. The method of claim 23, wherein the treatment comprises:
(a) treatment with durvalumab;
(b) treatment tremelimumab; or
(c) treatment with both durvalumab and tremelimumab.

25. (canceled)

26. The method of claim 23, wherein the head and neck cancer is a squamous cell carcinoma.

27. The method of claim 23, wherein the head and neck cancer is a recurrent cancer or a metastatic cancer.

28. (canceled)

29. A method of predicting success of head and neck cancer treatment in a patient in need thereof, comprising determining PD-L1 expression in the patient's tumor cells and tumor-associated immune cells, wherein ≥50% of tumor cells express PD-L1 and/or ≥25% of tumor-associated immune cells express PD-L1 predicts success of treatment.

30. The method of claim 29, wherein the treatment comprises:
(a) treatment with durvalumab;
(b) treatment tremelimumab; or
(c) treatment with both durvalumab and tremelimumab.

31. (canceled)

32. The method of claim 29, wherein the head and neck cancer is a squamous cell carcinoma.

33. The method of claim 29, wherein the head and neck cancer is recurrent or a metastatic cancer.

34. (canceled)

35. A method of treating head and neck cancer in a patient in need thereof, comprising:
(a) determining PD-L1 expression in the patient's tumor cells and tumor-associated immune cells; and
(b) treating or continuing treatment if ≥50% of the tumor cells express PD-L1 and/or ≥25% of the tumor-associated immune cells express PD-L1.

36. The method of claim 35, wherein the treatment comprises:
(a) treatment with durvalumab;
(b) treatment tremelimumab; or
(c) treatment with both durvalumab and tremelimumab.

37. (canceled)

38. The method of claim 35, wherein the head and neck cancer is a squamous cell carcinoma.

39. The method of claim 35, wherein the head and neck cancer is a recurrent cancer or a metastatic cancer.

40. (canceled)

41. A method of predicting success of head and neck cancer treatment in a patient in need thereof, comprising determining levels of one or a plurality of protein biomarkers, wherein the protein biomarker is IL-23, osteocalcin, IL-6, neutrophil-to-lymphocyte ratio (NLR), von Willebrand factor (vWF), or Plasminogen activator inhibitor-1 (PAI-1);
wherein an increased level of IL-23 or osteocalcin as compared to a reference level, and/or a decreased level of IL-6, NLR, vWF, or PAI-1 as compared to a reference level, and/or low tumor burden as compared to a reference level predicts success of treatment.

42. The method of claim 41, wherein the cancer treatment comprises treatment with durvalumab.

43. The method of claim 41, wherein the level of PAI-1 is <229 pg/mL, the level of IL-6 is <5.4 pg/mL, the level of IL-23 >is 2.1 pg/mL, and the level of osteocalcin is >32 pg/mL.

44. The method of claim 41, wherein the head and neck cancer is a squamous cell carcinoma.

45. The method of claim 41, wherein the head and neck cancer is a recurrent cancer or a metastatic cancer.

46. (canceled)

47. A method of treating head and neck cancer in a patient in need thereof, comprising:
(a) determining levels of one or a plurality of protein biomarkers, wherein the protein biomarker is IL-23, osteocalcin, IL-6, neutrophil-to-lymphocyte ratio (NLR), von Willebrand factor (vWF), or Plasminogen activator inhibitor-1 (PAI-1); and
(b) treating or continuing treatment if there is an increased level of IL-23 or osteocalcin as compared to a reference level, and/or a decreased level of IL-6, NLR, vWF, or PAI-1 as compared to a reference level, and/or low tumor burden as compared to a reference level.

48. The method of claim 47, wherein the cancer treatment comprises treatment with durvalumab.

49. The method of claim 47, wherein the level of PAI-1 is <229 pg/mL, the level of IL-6 is <5.4 pg/mL, the level of IL-23 >is 2.1 pg/mL, and the level of osteocalcin is >32 pg/mL.

50. The method of claim 47, wherein the head and neck cancer is a squamous cell carcinoma.

51. The method of claim 47, wherein the head and neck cancer is a recurrent cancer or a metastatic cancer.

52. (canceled)
